SEQUENCE LISTING (29 sequences)

SEQ ID NO: 1 - DNA, 20 bp, Artificial Sequence
  Outer Forward
acctcaccctgtggagccac

SEQ ID NO: 2 - DNA, 20 bp, Artificial Sequence
  Outer Reverse Primer
tcattcgtctgtttcccatt

SEQ ID NO: 3 - DNA, 20 bp, Artificial Sequence
  HBS Inner forward
atggtgcatctgactcctga

SEQ ID NO: 4 - DNA, 21 bp, Artificial Sequence
  HBS Inner reverse
cagtaacggcagacttctcca

SEQ ID NO: 5 - DNA, 30 bp, Artificial Sequence
  IVS 1-5 Inner primer
ctccttaaacctgtcttgtaaccttgttag

SEQ ID NO: 6 - DNA, 20 bp, Artificial Sequence
  IVS 1-5 Inner forward
tgaggccctgggcaggtagg

SEQ ID NO: 7 - DNA, 30 bp, Artificial Sequence
  Cd 41/42 Inner Reverse
gagtggacagatccccaaaggactcaacct

SEQ ID NO: 8 - DNA, 21 bp, Artificial Sequence
  Cd 41/42 Inner Forward
ccttggacccagaggttcatt

SEQ ID NO: 9 - DNA, 20 bp, Artificial Sequence
  Cd 15 Inner Reverse
atccacgttcaccttgcgcc

SEQ ID NO: 10 - DNA, 30 bp, Artificial Sequence
  Cd 15 inner forward
tgaggagaagtctgccgttactgcccagta

SEQ ID NO: 11 - DNA, 26 bp, Artificial Sequence
  Outer Forward 2
gtacggctgtcatcacttagacctca

SEQ ID NO: 12 - DNA, 20 bp, Artificial Sequence
  Cd 30 inner forward
ggtggtgaggccctggggag

SEQ ID NO: 13 - DNA, 30 bp, Artificial Sequence
  Cd 30 Inner Reverse
taaacctgtcttgtaaccttgatacctacg

SEQ ID NO: 14 - DNA, 28 bp, Artificial Sequence
  outer frward
caaggtgacagacaactggtgcaatgat

SEQ ID NO: 15 - DNA, 28 bp, Artificial Sequence
  outer reverse
agcccctgaaagcttcactacaggttcc

SEQ ID NO: 16 - DNA, 29 bp, Artificial Sequence
  ApoA5 IF
ttcagcttttcctcatggggcaaatatac

SEQ ID NO: 17 - DNA, 26 bp, Artificial Sequence
  ApoA5 IR
gagccccaggaactggagcgaaatta

SEQ ID NO: 18 - DNA, 26 bp, Artificial Sequence
  MTHFR IF
ttgaaggagaaggtgtctgcgggtgc

SEQ ID NO: 19 - DNA, 30 bp, Artificial Sequence
  MTHFR IR
caaagaaaagctgcgtgatgatgaaatgga

SEQ ID NO: 20 - DNA, 22 bp, Artificial Sequence
  CTRC X4 Forward
aaggacaatgggaacactctct

SEQ ID NO: 21 - DNA, 20 bp, Artificial Sequence
  CTRC X4 Reverse
tcaggtatggggtgcgacag

SEQ ID NO: 22 - DNA, 22 bp, Artificial Sequence
  SMN1ex7F
tttattttccttacagggtttc

SEQ ID NO: 23 - DNA, 19 bp, Artificial Sequence
  SMN1ex8RB
tggcctcccacccccaacc

SEQ ID NO: 24 - DNA, 24 bp, Artificial Sequence
  SMN1int7R
gtgaaagtatgtttcttccacgta

SEQ ID NO: 25 - DNA, 30 bp, Artificial Sequence
  MTHFR OF
tttgaggctgacctgaagcacttgaaggag

SEQ ID NO: 26 - DNA, 30 bp, Artificial Sequence
  MTHFR OR
gagtggtagccctggatgggaaagatcccg

SEQ ID NO: 27 - DNA, 25 bp, Artificial Sequence
  SMN1 i8F
ctggcatagagcagcactaaatgac

SEQ ID NO: 28 - DNA, 24 bp, Artificial Sequence
  TH6 F
gactcaaggctgagagatgcagga

SEQ ID NO: 29 - DNA, 24 bp, Artificial Sequence
  TH7 R
caatgtatcatgcctctttgcacc

(19) United States
(12) Patent Application Publication
(10) Pub. No.: US 2022/0372575 A1
(43) Pub. Date: Nov. 24, 2022

(54) A KIT FOR DETECTION OF MUTATIONS CAUSING GENETIC DISORDERS

(71) Applicant: COUNCIL OF SCIENTIFIC & INDUSTRIAL RESEARCH, New Delhi (IN)

(72) Inventors: Giriraj Ratan CHANDAK, Hyderabad (IN); Sumit PALIWAL, Hyderabad (IN); Swati BAYYANA, Hyderabad (IN); Vinay DONIPADI, Hyderabad (IN)

(21) Appl. No.: 17/763,340

(22) PCT Filed: Mar. 2, 2020
(86) PCT No.: PCT/IN2020/050189

§ 371 Date: Mar. 24, 2022

(30) Foreign Application Priority Data
Sep. 25, 2019 (IN) 201911038617

(51) Int. Cl. C12Q 1/6883

(52) U.S. Cl. CPC C12Q 1/6883; C12Q 1/6858

(57) ABSTRACT

The present invention is directed to a kit based on ARMS-PCR/AS-PCR in a single tube reaction for detection of mutations causing genetic disorders like hemoglobinopathies and musculopathies using unprocessed human dried blood spot.

### FIELD OF THE INVENTION

The present invention provides a kit for detection of mutations causing genetic disorders from unprocessed human dried blood spot using Amplification Refractory Mutation System (ARMS)/Allele-Specific (AS) Polymerase Chain Reaction (PCR) wherein the detection is performed in a single tube/reaction and a diagnostic kit thereof for detecting mutations that result in genetic disorders like hemoglobinopathies and musculopathies.

### BACKGROUND OF THE INVENTION

Over the last several years, genetic testing for a large number of genetic disorders including various hemoglobinopathies, musculopathies, neurodegenerative diseases, mitochondrial diseases, bleeding and clotting disorders is available. Genetic testing includes molecular diagnosis, carrier detection, predictive analysis, prenatal diagnosis, and genetic counselling. Hemoglobinopathies [including Beta thalassemia, Sickle cell anaemia, Haemoglobin E disease (HbE)] and Musculopathies [including Spinal Muscular Atrophy (SMA)], though rare, are one of the commonest single gene diseases and form bulk referrals for genetic testing. These diseases, caused by genetic variations (including point mutations, deletions, etc.) in a single gene, are readily identified since genetic testing in these diseases requires screening only one gene. Additionally, certain mutations are known to account for a large percentage of patients suffering from specific disease, e.g. one single mutation in the beta-globin gene accounts for all sickle cell anaemia patients, six common mutations in the beta-globin gene cause the disease in more than 95% of patients of Beta thalassemia, two deletions result in Spinal muscular atrophy in more than 95% patients, and deletions in specific regions of the dystrophin gene are found in close to 70% of all Duchenne muscular dystrophy (DMD) patients.

Various techniques for such genetic testing are available to detect mutations such as single nucleotide polymorphisms (SNPs), insertion-deletion mutations (InDels), etc. These include PCR-Restriction Fragment Length Polymorphism (PCR-RFLP), Multiplex PCR, Nested PCR, DNA sequencing, Allele-specific PCR, Amplification Refractory Mutation System (ARMS-PCR), Real-time based PCR, Reverse transcriptase PCR (RT-PCR), etc. Although they are specific but time-taking and expensive (1, 2). One of the reasons why they are time-taking because it requires sample collection, storage, transport and isolation of DNA from the blood samples. This also necessitates an infrastructure for this purpose. Alternate sources of template like whole blood, urine, saliva, etc. have been used earlier. For example, Factor V Leiden mutation and SNPs affecting one-carbon metabolism (includes folate and homocysteine metabolism) can be detected using whole blood ARMS-PCR to assess risk (6, 7). Detection of pathogens and pathogenic nucleic acids from dried blood also indicates a quick detection strategy (4). ARMS-PCR based detection using allele-specific primers for diseases/disorders are either real-time based or based on scorpion platform (3, 4). However, all these involve the step of pre-processing of the whole blood or dried blood spot (for generating a lysate or isolating genomic DNA) for subsequent PCR-based detection strategies thus making them time-consuming and expensive due to need for sophisticated instruments. Different types of polymerases have been involved in methods for directly amplifying nucleic acids from various biological samples as mentioned above or isolated genomic DNA etc. A mutated Taq polymerase for various purposes has been developed to detect various diseases using different PCR methods (8) (includes standard PCR, Real-time PCR, ARMS-PCR, etc.).

As explained above, there are established protocols and kits available for the detection of mutations resulting in genetic disorders, such as hemoglobinopathies and musculopathies; there are several challenges in genetic diagnosis such as isolation of DNA, transportation and storage of samples, sophisticated instruments to be run by technical experts, time-taking procedures and the high costs involved in detecting and diagnosing them with specificity. It would, therefore, be imperative to devise new methods and kits for detecting the genetic diseases as mentioned earlier which could overcome the challenges listed above, and which would especially be time and cost-efficient in identifying these genetic diseases. The major aim of the present invention is to develop simple and affordable methods for the detection of mutations causing various genetic disorders such as hemoglobinopathies and musculopathies.

### OBJECTIVES OF THE INVENTION

The major objective of the invention is using unprocessed human dried blood spot (DBS) spotted on whattman filter paper for detection of mutations causing genetic disorders.

The another objective of the invention is providing a method of ARMS-PCR/AS-PCR in a single tube using unprocessed human dried blood spot for the detection of genetic diseases like hemoglobinopathies (including sickle cell anaemia, beta thalassemia, HbE disease) and musculopathies [like spinal muscular atrophy (SMA)].

Another major objective of the invention is providing the synthetic oligonucleotides used for the detection of above genetic disorders.

Another major objective of the invention is providing specific PCR conditions used for the detection of six common mutations causing hemoglobinopathies.

Another major objective of the invention is providing the synthetic oligonucleotides and specific PCR conditions used for the detection of spinal muscular atrophy.

Yet the another major objective of the invention is directed to developing a diagnostic method which is time-efficient and cost-effective to detect mutations causing single gene disorders like hemoglobinopathies and SMA. Also, this invention can be further extended to other genetic and complex disorders.

### SUMMARY OF THE INVENTION

Accordingly, the present invention is directed toward the use of unprocessed human dried blood spot as the sample for detection of mutations resulting in single gene disorders.

Another embodiment of the invention is an ARMS-PCR kit for detection of mutations causing single gene disorder consisting of:
a) Primers, having SEQID NO. 1-13,28 &29 for hemoglobinopathies
b) Primers, having SEQID NO. 20-24 &27 for spinal muscular atrophy
c) PCR reagents

In yet another embodiment, the in-vitro method for detection of single gene disorders using the kit.

In yet another embodiment, the thermal cycling conditions of the PCR amplification for detection of mutations resulting in hemoglobinopathies is selected from:

| NUMBER OF CYCLES | TEMPERATURE | TIME | |
|---|---|---|---|
| Denaturation-1 cycle | 95° C. | 3 | minutes |
| Annealing - 35 cycles | 95° C. | 20 | seconds |
| (Touch-down) | 65° C.-0.2° C. | 30 | seconds |
| | 68° C. | 1.5 | minutes |
| Extension -1 cycle | 68° C. | 10 | minutes |

In yet another embodiment, the thermal cycling conditions of the PCR amplification for detection of deletion mutations causing spinal muscular atrophy is selected from:

| NUMBER OF CYCLES | TEMPERATURE | TIME | |
|---|---|---|---|
| Denaturation-1 cycle | 95° C. | 3 | minutes |
| Annealing - 35 cycles | 95° C. | 20 | seconds |
| (Touch-down) | 65° C.-0.2° C. | 30 | seconds |
| | 68° C. | 1 | minute |
| Extension -1 cycle | 68° C. | 10 | minutes |

In another embodiment, use of the kit for in-vitro diagnostics of mutations causing hemoglobinopathies.

In another embodiment, use of the kit for in-vitro diagnostics of deletion mutations resulting in spinal muscular atrophy.

### BRIEF DESCRIPTION OF THE ACCOMPANYING DRAWINGS

FIG. 1 is a schematic representation to explain the basic principle of Amplification Refractory Mutation System-Polymerase chain reaction [ARMS -PCR]. The picture represents a hypothetical DNA sequence AB where M is the target mutation. A non-allele-specific control amplicon is amplified by 2 common (outer) primers [OF & OR] flanking the mutation. Two allele-specific (inner) primers are designed in opposite orientation to the common primers; wild-type forward (WtF) and mutant reverse (MutR). In combination with the common primers, inner primers amplify both wild and mutant alleles; OF and MutR amplify mutant allele and WtF and OR amplify wild allele. The control amplicon provides an internal control with respect to PCR amplification.

FIG. 2 represents the migration pattern of DNA on a 2% agarose gel used to distinguish three genotypes of Cd 6 (A>T) mutation in HBB gene causing Sickle cell anaemia. From left, lane 1: DNA marker, lane 2: wild (AA), lane 3: heterozygous (AT), lane 4: mutant (TT) and lane 5: negative control. The gel was run at 80 volts for 1 hour. Presence of control band in lane 2, 3 and 4 confirms PCR amplification.

FIG. 3A represents the migration pattern of DNA on 2% agarose gel used to distinguish various genotypes of IVS 1-5(G>C) mutation in HBB gene causing Beta-thalassemia. From left, lane 1: DNA marker, lane 2: wild (GG), lane 3: heterozygous (GC), lane 4: mutant (CC) and lane 5: negative control. The gel was run at 80 volts for 1 hour. Presence of control band in lane 2, 3 and 4 confirms PCR amplification.

FIG. 3B represents the migration pattern of DNA on 2% agarose gel used to distinguish various genotypes of Cd 41/42 (-CTTT) mutation in the HBB gene causing Beta-thalassemia. From left lane 1: DNA marker, lane 2: wild, lane 3: heterozygous, lane 4: mutant and lane 5: negative control. The gel was run at 80 volts for 1 hour. Presence of control band in lane 2, 3 and 4 confirms PCR amplification.

FIG. 3C represents the migration pattern of DNA on 2% agarose gel used to distinguish various genotypes of Cd15 (G>A) mutation in the HBB gene causing Beta-thalassemia. From left lane 1: DNA marker, lane 2: wild (GG), lane 3: heterozygous (GA), lane 4: mutant (AA) and lane 5: negative control. The gel was run at 80 volts for 1 hour. Presence of control band in lane 2, 3 and 4 confirms PCR amplification.

FIG. 3D represents the migration pattern of DNA on 2% agarose gel used to distinguish various genotypes of Cd30 (G>C) mutation in the HBB gene causing Beta-thalassemia. From left, lane 1: DNA marker, lane 2: wild (GG), lane 3: heterozygous (GC) and lane 4: negative control. The gel was run at 80 volts for 1 hour. Presence of control band in lane 2 and 3 confirms PCR amplification.

FIG. 4 represents the migration pattern of DNA on 2% agarose gel for five common mutations in HBB gene causing of Beta-thalassemia. A DBS sample has been tested for 5 mutations using a common PCR protocol. From left, lane 1: DNA marker, lane 2: heterozygous for IVS 1-5(G>C), lane 3: normal for Cd 41/42 (-CTTT) deletion, lane 4: normal for Cd 15 (G>A), lane 5: normal for Cd 30 (G>C), lane 6: normal for 619 bp deletion and lane 7: negative sample. ‘C’ represents control band, ‘W’ and ‘M’ represent wild and mutant alleles respectively.

FIG. 5 represents the migration pattern of DNA on 2% agarose gel for exon 7 and 8 deletions in the SMN gene causing Spinal muscular atrophy. From left, lane 1: DNA marker, lane 2: both exons (7 and 8) deleted, lane 3: exon 7 deleted, lane 4: wild and lane 5: negative control. The gel was run at 80 volts for 1 hour. Presence of control band in lane 2, 3 and 4 confirms PCR amplification.

FIG. 6 represents the migration pattern of DNA on 2% agarose gel used to distinguish various genotypes of mutation −1131 T>C in APOA5 gene associated with plasma triglycerides levels. From left lane 1: DNA marker, lane 2: heterozygous (TC), lane 3: heterozygous (TC), lane 4: wild (TT), lane 5: wild (TT), lane 6: heterozygous (TC) and lane 7: negative control. The gel was run at 80 volts for 1 hour. Presence of control band in lane 2-6 confirms PCR amplification.

FIG. 7 represents the migration pattern of DNA on 2% agarose gel used to distinguish various genotypes of mutation 677 C>T in MTHFR gene associated with plasma homocysteine levels. From left, lane 1: DNA marker, lane 2: wild (CC), lane 3: heterozygous (CT), lane 4: mutant (TT) and lane 5: negative control. The gel was run at 80 volts for 1 hour. Presence of control band in lane 2, 3 and 4 confirms PCR amplification.

### DETAILED DESCRIPTION OF THE INVENTION

The present invention is directed to a simple and affordable protocol for rapid detection of mutations associated with single gene disorders, mainly hemoglobinopathies and musculopathies. The ARMS-PCR method developed in the present invention uses unprocessed human dried blood spot spotted on Whatman filter paper as the template. A tetra-primer amplification refractory mutation system (ARMS-PCR) amplifies both wild-type and mutant alleles, together with a control fragment, in a single tube reaction (FIG. 1). Primers referred to in this invention are synthetic oligonucleotides which are specifically designed and chemically synthesized in vitro.

The first embodiment of the present invention is a method for detection of mutations causing genetic disorders by ARMS-PCR technique wherein the ARMS-PCR reaction is carried out in a single tube using unprocessed human dried blood spot as template.

Another embodiment of the present invention is a method for detection of mutations causing genetic disorders of the type of hemoglobinopathies (including Sickle cell anaemia, Beta-thalassemia) and musculopathies (including Spinal muscular atrophy) by ARMS-PCR in a single tube, wherein the template used is unprocessed human dried blood spot. The term “template” means source of DNA which is to be analysed or amplified.

Another embodiment of the present invention is a method for detection of mutations causing genetic disorders of the type of hemoglobinopathies (including Sickle cell anaemia, Beta-thalassemia) and musculopathies (including Spinal muscular atrophy) by ARMS-PCR in a single tube, wherein the template used is unprocessed human whole blood.

Yet another embodiment of the invention detects mutations like single nucleotide polymorphisms, frameshift mutations, insertions and deletions using the ARMS-PCR reaction of this invention.

One other embodiment of the present invention is a method of ARMS-PCR wherein the steps for PCR amplification protocol for detecting hemoglobinopathies like Sickle cell anemia and Beta thalassemia include the steps of (a) an initial denaturation cycle of 95° C. for 3 mins, (b) amplification cycle of 95° C. for 20 secs, 65° C.−0.2° C. [Touch-down PCR] for 30 secs and 68° C. for 1.5 mins for 35 cycles, and (c) extension at 68° C. for 10 mins.

Yet another embodiment of the present invention makes use of synthetic oligonucleotides for the detection of Sickle cell anaemia and Beta thalassemia which are selected from the group of synthetic oligonucleotides of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9, SEQ ID NO. 10, SEQ ID NO. 11, SEQ ID NO. 12, SEQ ID NO. 13, SEQ ID NO. 28 and SEQ ID NO. 29.

One more embodiment of the current invention provides a method which detects the wild-type, mutant and heterozygote genotypes of each mutation in the beta-globin gene in a single tube PCR reaction.

Another embodiment of the current invention provides a method for the detection of wild-type, mutant and heterozygote genotypes of common mutations in the beta-globin gene is performed together in a single PCR condition.

In another embodiment of the invention, the method of ARMS-PCR reaction for detecting Spinal muscular atrophy includes the steps of: (a) an initial denaturation cycle of 95° C. for 3 mins, (b) amplification cycle of 95° C. for 20 secs, 60° C. for 30 secs and 68° C. for 1 min for 35 cycles, and (c) extension at 68° C. for 10 mins.

In yet another embodiment of this invention, the synthetic oligonucleotides used for the detection of Spinal muscular atrophy are selected from the group of synthetic oligonucleotides of SEQ ID NO.20, SEQ ID NO. 21, SEQ ID NO. 22, SEQ ID NO. 23, SEQ ID NO. 24 and SEQ ID NO. 27.

In one more embodiment of the invention, the deletion mutations of exon 7 and exon 8 of SMN gene are identified in single tube PCR reaction. In a further embodiment of the invention, the method of identifying deletion mutations of exon 7 and exon 8 of SMN gene simultaneously distinguishes between SMN1 and SMN2 copies of SMN gene.

One more embodiment of the present invention is a diagnostic kit for detecting single nucleotide polymorphisms, multiple mutations, insertions and deletions causing hemoglobinopathies using unprocessed human dried blood spot comprising (i) Synthetic oligonucleotides of SEQ ID numbers 1, 2, 3, 4, 5, 6, 7, 8, 9, 10, 11, 12, 13, 20, 28 and 29, (ii) PCR master mix containing dNTPs, MgCl₂ and PCR buffer, and (iii) a Taq polymerase.

A further embodiment of the present invention is a diagnostic kit for detecting multiple deletions causing spinal muscular atrophy using unprocessed human dried blood spot comprising (i) Synthetic oligonucleotides of SEQ ID numbers 20, 21, 22, 23, 24 and 27, (ii) PCR master mix containing dNTPs, MgCl₂ and PCR buffer, and (iii) a Taq polymerase.

In another embodiment of the invention, the method of ARMS-PCR for detecting Sickle cell anaemia (SCA), a type of hemoglobinopathies is based on synthetic oligonucleotides selected from the group of artificial nucleotides of SEQ ID NO. 1, SEQ ID NO 2, SEQ ID NO 3 and SEQ ID NO 4.

In yet another embodiment of the invention, the method of ARMS-PCR for detecting Beta-thalassemia, a type of hemoglobinopathies is based on synthetic oligonucleotides selected from the group of artificial nucleotides of SEQ ID NO. 1, SEQ ID NO 2, SEQ ID NO 5, SEQ ID NO 6, SEQ ID NO 7, SEQ ID NO 8, SEQ ID NO 9, SEQ ID NO 10, SEQ ID NO 11, SEQ ID NO 12, SEQ ID NO 13, SEQ ID NO 28 and SEQ ID NO 29.

In one embodiment of the invention, five or more common mutations (IVS 1-5 G>C, Cd 41/42-CTTT, Cd 15 G>A, Cd 30 G>C, 619 bp deletion and HbS Cd6 T>A) in the beta-globin gene (HBB) causing hemoglobinopathies can be detected together in a single ARMS-PCR condition.

A further embodiment of the present invention is the use of synthetic oligonucleotides of SEQ ID Numbers 1, 2, 3, 4, 5, 6, 7, 8, 9, 10, 11, 12, 13, 28 and 29 for the identification and detection of hemoglobinopathies.

Another embodiment of the invention is the use of synthetic oligonucleotides of SEQ ID numbers 20, 21, 22, 23, 24 and 27 for the identification and detection of Spinal muscular atrophy.

A further embodiment of this invention has the synthetic oligonucleotides of SEQ ID numbers 1-29.

The present invention discloses an ARMS-PCR method specifically suitable for quick diagnosis of hemoglobinopathies and musculopathies from unprocessed human dried blood spot in a single tube reaction which overcomes all the earlier described challenges.

### EXAMPLES

The following examples are given by way of illustration of the present invention and therefore should not be construed to limit the scope of the present invention.

### Example 1

ARMS-PCR assay for detection of mutation causing Sickle Cell Anaemia using unprocessed human dried blood spot (DBS) in a single reaction.

To test the ability of the method developed in the present invention, unprocessed human dried blood spot were used. Multiple primers were designed to detect wild-type genotype (A/A), mutant genotype (T/T) and heterozygote genotype (A/T) at Cd6 T>A mutation in HBB gene. An outer forward primer [50 nM, 20 nt, 5′ d(ACC TCA CCC TGT GGA GCC AC) 3′] (SEQ ID NO:1) and outer reverse primer [50 nM, 20 nt, 5′ d(TCA TTC GTC TGT TTC CCA TT) 3′] (SEQ ID NO: 2), allele-specific primers, inner forward for A allele [50 nM, 20 nt, 5′ d(ATG GTG CAT CTG ACT CCT GA) 3′] (SEQ ID NO:3) and inner reverse for T allele [50 nM, 21 nt, 5′ d(CAG TAA CGG CAG ACT TCT CCA) 3′] (SEQ ID NO:4) were used for detection of specific allele. Templates for all the three genotypes (mutant, heterozygote and wild-type) were used. Reaction mixtures contained 1X buffer including 60 mM Tricine, 5 mM (NH₄)₂SO₄, 3.5 mM MgCl₂, 6% glycerol, pH −8.7, 2.5 mM of each dNTP and 0.5 volumes of HemoKlenTaq for unprocessed human dried blood spot sample. Preceding the reaction, the unprocessed human dried blood spot equivalent to 1 ul which was spotted previously was cut and added to the reaction tube. After an initial denaturation cycle (95° C. for 3 mins), the product was amplified for 35 cycles (95° C. for 20 secs, 65° C. −0.2° C. [Touch-down PCR] for 30 secs and 68° C. for 1.5 mins) and final extension at 68° C. for 10 mins. Finally, the PCR products were analysed on 2% agarose gel and result interpreted.

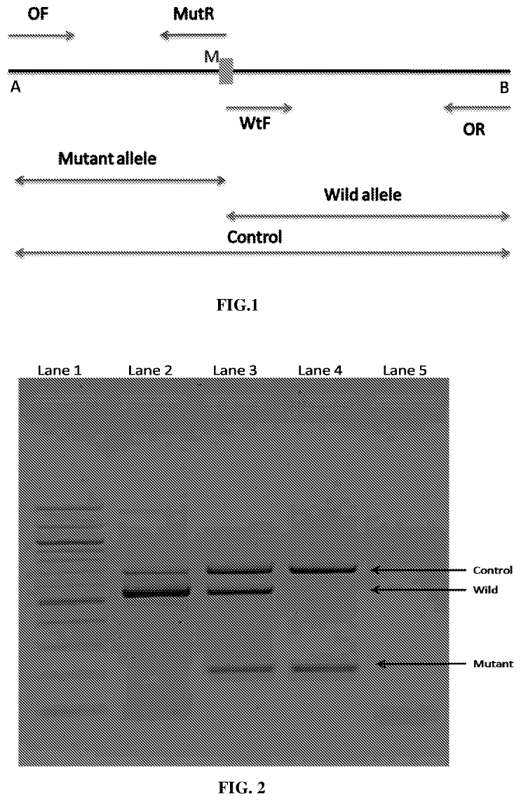

The method was successfully tested on all templates, displaying a wild-type, mutant and heterozygote genotype. As depicted in FIG. 2, all three possible SNP genotypes (showing various combinations of wild and mutant alleles) in sickle cell anaemia can be clearly distinguished. Therefore, fast and reliable SNP genotyping is possible using the present method, thus concluding that direct detection of SNPs from unprocessed human dried blood spot samples is achievable using the single tube PCR method developed.

### Example 2

ARMS-PCR assay for detection of 5 common mutations causing Beta-thalassemia using unprocessed human dried blood spot in a single PCR condition individually.

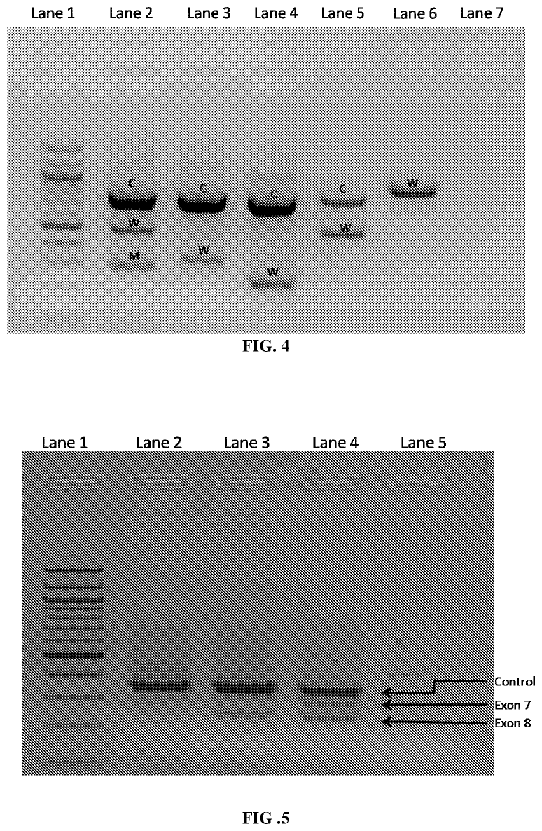

An ARMS-PCR assay for Beta-Thalassemia mutations using unprocessed human dried blood spot in one common PCR condition was established as depicted in FIG. 4. Allele-specific primers have been used so that both the alleles can be detected in the same reaction. To detect the wild-type, mutant and heterozygote genotypes, a number of primers have been designed. An outer forward primer1 [50 nM, 20 nt, 5′ d(ACC TCA CCC TGT GGA GCC AC) 3′] (SEQ ID NO:1), an outer forward primer2 [50 nM, 26 nt, 5′ d(GTA CGG CTG TCA TCA CTT AGA CCT CA) 3′] (SEQ ID NO:11) and an outer reverse primer [50 nM, 20 nt, 5′ d(TCA TTC GTC TGT TTC CCA TT) 3′] (SEQ ID NO:2) were used. For IVS1>5 G>C mutation in HBB gene, allele specific primers inner forward for G [50 nM, 20 nt, 5′ d(TGA GGC CCT GGG CAG GTA GG) 3′] (SEQ ID NO:6) and inner reverse for C [50 nM, 30 nt, 5′ d(CTC CTT AAA CCT GTC TTG TAA CCT TGT TAG) 3′] (SEQ ID NO:5); for Cd 41/42 -CTTT deletion mutation in HBB gene, specific inner primer forward [50 nM, 21 nt, 5′ d(CCT TGG ACC C AG AGG TTC ATT) 3′] (SEQ ID NO:8) and inner reverse primer [50 nM, 30 nt, 5′ d(GAG TGG ACA GAT CCC CAA AGG ACT CAA CCT) 3′] (SEQ ID NO:7); for Cd15 G>A mutation in HBB gene, allele specific primer inner forward for G [50 nM, 30 nt, 5′ d(TGA GGA GAA GTC TGC CGT TAC TGC CCA GTA) 3′] (SEQ ID NO:10) and inner reverse for A [50 nM, 20 nt, 5′ d(ATC CAC GTT CAC CTT GCG CC) 3′] (SEQ ID NO:9); for Cd 30 G>C mutation in HBB gene, allele specific primer inner forward for G [50 nM, 20 nt, 5′ d(GGT GGT GAG GCC CTG GGG AG) 3′] (SEQ ID NO:12) and inner reverse for C [50 nM, 30 nt, 5′ d(TAA ACC TGT CTT GTA ACC TTG ATA CCT ACG) 3′] (SEQ ID NO:13); for 619 bp deletion in HBB gene forward primer [50 nM, 24 nt, 5′d(GAC TCA AGG CTG AGA GAT GCA GGA) 3′] (SEQ ID NO:28) and reverse primer [50 nM, 24 nt, 5′d(CAA TGT ATC ATG CCT CTT TGC ACC) 3′] (SEQ ID NO:29) were used. Templates for mutant, heterozygote and wild-type genotypes were used. Reaction mixtures contained 1X buffer including 60 mM Tricine, 5 mM (NH₄)₂SO₄, 3.5 mM MgCl₂, 6% glycerol, pH −8.7, 2.5 mM of each dNTP and 0.5 volumes of HemoKlenTaq for unprocessed human dried blood spot. Preceding the reaction, the unprocessed human dried blood spot equivalent to 1 ul which was spotted previously was cut and added to the reaction tube. After an initial denaturation cycle (95° C. for 3 mins), the product was amplified for 35 cycles (95° C. for 20 secs, 65° C. −0.2° C. [Touch-down PCR] for 30 secs and 68° C. for 1.5 mins) and final extension was performed at 68° C. for 10 mins Finally, the PCR products were analysed by 2% agarose gel electrophoresis and the results interpreted.

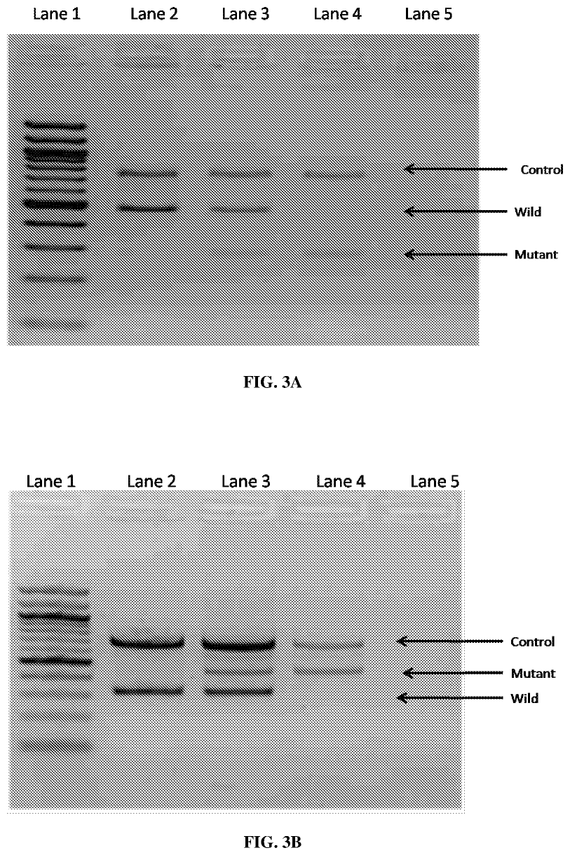

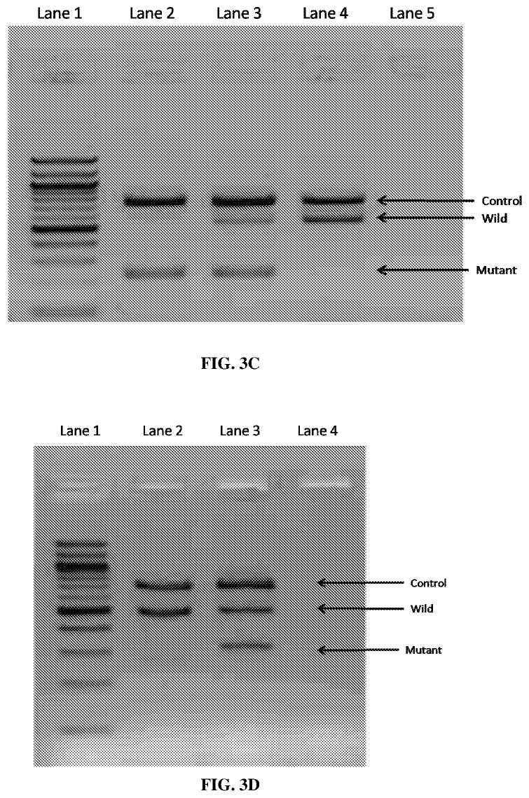

The method was successfully tested on all templates, displaying a wild-type, mutant and heterozygote genotype as exemplified in FIGS. 3A-3D. As inferred from FIG. 4, all three possible SNP genotypes for individual mutations (IVS 1-5 G>C, Cd 41/42-CTTT, Cd 15 G>A and Cd30 G>C mutation) were clearly detected in one reaction and all the common 5 mutations were detected in one PCR condition. Therefore, the direct detection of the 5 common SNPs causing Beta-Thalassemia is achievable from unprocessed human dried blood spot in a single PCR reaction.

### Example 3

Allele-specific PCR assay for detection of deletions of exons causing Spinal Muscular Atrophy (SMA) using unprocessed human DBS in a single reaction.

To detect the deletions in the SMN gene for the diagnosis of SMA using unprocessed human dried blood spot, multiple allele-specific primers were designed. This would detect the deletion of exon 7 and exon 8 in the SMN gene and simultaneously distinguish between SMN1 and SMN2 copy of the SMN gene in one single reaction. A forward primer for exon 7 [50 nM, 22 nt, 5′ d(TTT ATT TTC CTT ACA GGG TTT C) 3′] (SEQ ID NO:22), an inner reverse primer [50 nM, 24 nt, 5′ d(GTG AAA GTA TGT TTC TTC CAC GTA) 3′] (SEQ ID NO:24), an inner forward primer for exon 8 [50 nM, 25 nt, 5′ d(CTG GCA TAG AGC AGC ACT AAA TGA C) 3′] (SEQ ID NO:27), reverse primer specific for exon8 of SMN1 [50 nM, 19 nt, 5′ d((TGG CCT CCC ACC CCC AAC C) 3′] (SEQ ID NO:23) were used. As an internal control, a control forward primer [50 nM, 22 nt, 5′d (AAG GAC AAT GGG AAC ACT CTC T) 3′] (SEQ ID NO: 20) and a control reverse primer [50 nM, 20 nt, 5′d (TCA GGT ATG GGG TGC GAC AG) 3′] (SEQ ID No: 21) were also used in the same reaction. Templates for exon 7 deletion, exon 8 deletion and both exons 7 and 8 deletions in the SMN1 copy of the SMN gene were used to validate the method. Reaction mixtures contained 1X buffer (including 60 mM Tricine, 5 mM (NH₄)₂SO₄, 3.5 mM MgCl₂, 6% glycerol, pH −8.7), additional 1.5 mM MgCl₂, 2.5 mM of each dNTP and 0.5 volumes of HemoKlenTaq for unprocessed human dried blood spot. Preceding the reaction, the unprocessed human dried blood spot equivalent to 1 ul which was spotted previously was cut and added to the reaction tube. After an initial denaturation cycle (95° C. for 3 mins), the product was amplified for 35 cycles (95° C. for 20 secs, 60° C. for 30 secs and 68° C. for 1 min) and a final extension at 68° C. for 10 mins Finally, the PCR products were analysed on 2% agarose gel and results interpreted as depicted in FIG. 5.

The assay was successfully tested on all templates, displaying exon 7 deletions, exon 8 deletions and both exon deletion in the SMN1 copy of the SMN gene. As shown in FIG. 5, for both exon 7 deletion and exon 8 deletion the product shows control band along with non-deleted exon band respectively. As depicted in FIG. 5, deletion of both exons 7 & 8 of SMN1 gene can be clearly identified and both SMN1 and SMN2 copies can be distinguished using unprocessed human DBS sample.

### Example 4

ARMS-PCR assay for detection of a mutation (SNP) in APOA5 gene using unprocessed human DBS in a single reaction.

A total of four primers were designed to detect wild-type genotype (T/T), mutant genotype (C/C) and heterozygote genotype (T/C) for APOA5 gene using the ARMS-PCR method in the present invention.

An outer forward primer [50 nM, 28 nt, 5′ d(CAA GGT GAC AGA CAA CTG GTG CAA TGA T) 3′] (SEQ ID NO:14), an outer reverse primer [50 nM, 28 nt, 5′ d(AGC CCC TGA AAG CTT CAC TAC AGG TTC C) 3′] (SEQ ID NO:15), allele-specific primers inner forward for the T allele[50 nM, 29 nt, 5′ d(TTC AGC TTT TCC TCA TGG GGC AAA TAT AC) 3′] (SEQ ID NO:16) and inner reverse for the C allele [50 nM, 26 nt, 5′ d(GAG CCC CAG GAA CTG GAG CGA AAT TA) 3′] (SEQ ID NO:17) were used. Appropriate templates for the mutant, heterozygote and wild-type genotypes were used. Reaction mixtures contained 1X buffer including 60 mM Tricine, 5 mM (NH₄)₂SO₄, 3.5 mM MgCl₂, 6% glycerol, pH −8.7, 2.5 mM of each dNTP and 0.5 volumes of HemoKlenTaq. Preceding the reaction, the unprocessed human dried blood spot equivalent to 1 ul which was spotted previously was cut and added to the reaction tube. After an initial denaturation cycle (95° C. for 3 mins), the product was amplified for 35 cycles (95° C. for 45 secs, 59° C. for 45 secs and 68° C. for 1 min) and a final extension at 68° C. for 10 min. Finally, the PCR products were analysed on 2% agarose gel and result interpreted.

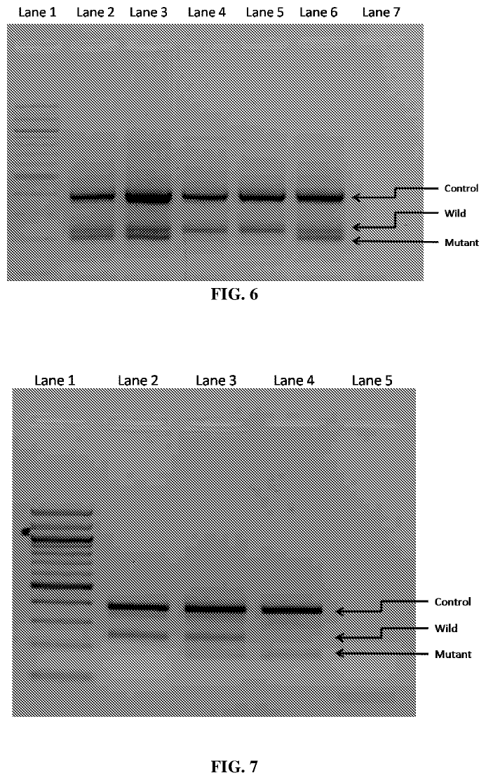

The assay was successfully tested on all templates, displaying a wild-type, mutant and heterozygote genotype, as depicted in FIG. 6. Thus, all three genotypes of APOA5 gene associated with triglyceride metabolism can be easily, quickly and reliably detected through the single tube PCR method developed in the present invention.

### Example 5

ARMS-PCR assay for detection of a mutation (SNP) in MTHFR gene using unprocessed human DBS in a single reaction.

Multiple primers were designed to detect the C677T mutation in the MTHFR gene using the ARMS-PCR method developed in the present invention utilizing the unprocessed human DBS.

An outer forward primer [50 nM, 30 nt, 5′ d(TTT GAG GCT GAC CTG AAG CAC TTG AAG GAG) 3′] (SEQ ID NO:25), an outer reverse primer [50 nM, 0 nt, 5′ d(GAG TGG TAG CCC TGG ATG GGA AAG ATC CCG) 3′] (SEQ ID NO:26) and allele-specific primers inner forward for the C allele [50 nM, 26 nt, 5′ d(TTG AAG GAG AAG GTG TCT GCG GGT GC)3′](SEQ ID NO:18) and inner reverse for the T allele [50 nM, 30 nt, 5′ d(CAA AGA AAA GCT GCG TGA TGATGA AAT GGA) 3′] (SEQ ID NO:19) were used. Templates for mutant, heterozygote and wild-type genotypes were used. Reaction mixtures contained 1X buffer including 60 mM Tricine, 5 mM (NH₄)₂SO₄, 3.5 mM MgCl₂, 6% glycerol, pH −8.7, 2.5 mM of each dNTP and 0.5 volumes of HemoKlenTaq. Preceding the reaction, the unprocessed human dried blood spot equivalent to 1 ul which was spotted previously was cut and added to the reaction tube. After an initial denaturation cycle (95° C. for 3 mins), the product was amplified by 35 cycles (95° C. for 20 secs, 65° C. -0.2° C. [Touch-down PCR] for 30 secs and 68° C. for 1.5 mins) and final extension at 68° C. for 10 mins. Finally, the PCR products analysed on 2% agarose gel and results interpreted.

The assay was successfully tested on all templates, displaying a wild-type, mutant and heterozygote genotype. As depicted in FIG. 7, all three possible SNP genotypes for MTHFR gene were clearly distinguished using PCR method developed in the present invention. Therefore, fast and reliable SNP genotyping using unprocessed human dried blood spot samples is achievable in the present invention.

The present invention uses unprocessed human dried blood spot (DBS) directly in the PCR reaction. The present invention uses specifically designed allele-specific/ARMS primers for specific disorders in the PCR reaction. The present invention detects several mutations resulting in Beta-thalassemia in a single reaction and under the same PCR conditions. The present invention detects the deletions causing spinal muscular atrophy in a single reaction. Diagnosis of genetic disorders can be done within a few hours (3-4 hrs) of collecting the sample; hence the protocol is time-efficient. The identification and diagnosis method of the invention is cost-effective as it utilizes a minimal amount of each PCR component thus significantly reducing the diagnostic testing costs.

### ADVATAGES OF THE INVENTION

The present invention provides:
1. A simple and affordable kit for rapid detection of mutations causing single gene disorders, mainly hemoglobinopathies (including sickle cell anemia and beta thalassemia) and musculopathies (including spinal muscular atrophy).
2. The protocol uses unprocessed human dried blood spot (DBS) spotted on Whatman filter paper as the template.
3. The ARMS-PCR/AS-PCR method of the present invention can detect multiple mutations.
4. In the present invention synthetic oligonucleotides have been used to detect hemoglobinopathies and musculopathies using the method of ARMS-PCR from unprocessed human dried blood spot (DBS).
5. The present invention is also directed to developing a diagnostic method which is time-efficient as detection of mutations can be completed within 3 hours.
6. It would only cost between Rs 20-25 per sample, which may get even lower if multiple samples are used simultaneously.

### REFERENCES

1. Diagnosis and treatment of SMA and SMN deficiency. Patent no. US2015025817A1. Brian D. Mccabe and LIvioPellizzoni. Columbia University of New York. Oct. 10, 2012.

2. Detection, identification, validation and enrichment of target nucleic acids. Patent no. WO2016053883A1. Edgar Schreiber, Kamini Varma and Mark Andersen. Life Technologies Corporation. Oct. 3, 2014.

3. Methods for nucleic acid amplification. Patent no. WO201566530. Chen-HsiungYeh. Atherotech, Inc. Oct. 31, 2013.

4. Methods for amplifying and detecting pathogen nucleic acid in a whole blood sample. Patent no. EP3312640A1. Thomas Jay Lowery et al. T2 biosystems Inc. Oct. 22, 2010.

5. Use of whole blood in PCR reactions. U.S. Pat. No. 7,462,475B2. Milko B. Kermekchiev and Wayne M. Barnes. DNA Polymerase Technology Inc. May 20, 2004.

6. Development of a rapid DNA screening procedure for the Factor V Leiden mutation. Scobie GA, Ho ST, Dolan G, Kalsheker NA. Clinical Molecular Pathology. 1996 December, 49(6):M361-3. PMID—16696104.

7. Single Nucleotide Polymorphism (SNP) genotyping in unprocessed whole blood and serum by Real-Time PCR: Application to SNPs affecting Homocysteine and

Folate metabolism. Ulvik A and Ueland PM. Clinical Chemistry. 2001 November; 47(11):2050-3. PMID—11673381

8. Mutated DNA Polymerases with High selectivity and activity. Patent no. US20160298174A1. Andreas Marx, Matthias Drum and Ramon Kranaster. Universitaet Konstanz. Dec. 2, 2013.

1-7. (canceled)

8. A kit for rapid detection of mutations causing genetic disorders from an unprocessed human blood sample in a single tube reaction, the kit comprising:
(a) a first group of primers for the detection of mutations causing hemoglobinopathies, the first group of primers having the sequences set forth in SEQ ID NOS. 1-13, 28, and 29;
(b) a second group of primers for the detection of mutations causing spinal muscular atrophy, the second group of primers having the sequences set forth in SEQ ID NOS. 20-24 and 27; and
(c) PCR reagents.

9. The kit of claim 8, wherein the sample used for detection is an unprocessed dried blood spot (DBS).

10. The kit of claim 8, wherein the sample used for detection is an unprocessed whole blood sample.

11. The kit of claim 8, wherein the rapid detection of mutations is performed using a tetra-primer based Amplification Refractory Mutation System (ARMS) PCR method.

12. The kit of claim 8, wherein the kit detects homozygous wild genotypes, homozygous mutant genotypes, and heterozygous genotypes in the single tube reaction.

13. The kit of claim 8, wherein the rapid detection of mutations causing hemoglobinopathies using PCR is obtainable from the kit by a thermal cycling comprising:
a denaturation cycle at 95° C. for 3 minutes;
thirty-five annealing cycles, each annealing cycle comprising:
a first phase at 95° C. for 20 seconds;
a touchdown phase for 30 seconds, wherein a temperature of the touchdown phase is 65° C. for a first annealing cycle of the thirty-five annealing cycles and is decreased by 0.2° C. for each successive annealing cycle; and
a final phase at 68° C. for 1.5 minutes; and
an extension cycle at 68° C. for 10 minutes.

14. The kit of claim 8, wherein the rapid detection of mutations causing spinal muscular atrophy using PCR is obtainable from the kit by a thermal cycling comprising:
a denaturation cycle at 95° C. for 3 minutes;
thirty-five annealing cycles, each annealing cycle comprising:
a first phase at 95° C. for 20 seconds;
a second phase at 60° C. for 30 seconds; and
a third phase, at 68° C. for 1 minute; and
an extension cycle at 68° C. for 10 minutes.

15. The kit of claim 14, wherein the mutations causing spinal muscular atrophy are a deletion of exon 7 in an SMN gene and a deletion of exon 8 in the SMN gene.

16. The kit of claim 15, wherein the instructions further comprise simultaneous detection of the deletion of exon 7, the deletion of exon 8, and discrimination between an SMN1 copy of the SMN gene and an SMN2 copy of the SMN gene.

17. The kit of claim 8, wherein the mutations detected are substitutions, frameshift mutations, insertions, deletions, and indels.

18. A method for using the kit according to claim 8 for simultaneous in-vitro detection of at least five mutations causing hemoglobinopathies, the method comprising:
adding an unprocessed human blood sample to a reaction tube comprising the first group of primers and the PCR reagents from the kit;
thermal cycling the reaction tube using PCR, the thermal cycling comprising:
a denaturation cycle at 95° C. for 3 minutes;
thirty-five annealing cycles, each annealing cycle comprising:
a first phase at 95° C. for 20 seconds;
a touchdown phase for 30 seconds, wherein a temperature of the touchdown phase is 65° C. for a first annealing cycle of the thirty-five annealing cycles and is decreased by 0.2° C. for each successive annealing cycle; and
a final phase at 68° C. for 1.5 minutes; and
an extension cycle at 68° C. for 10 minutes.

19. A method for using the kit according to claim 8 for simultaneous in-vitro detection of a deletion of exon 7 in an SMN gene and a deletion of exon 8 in the SMN gene and discrimination between an SMN1 copy of the SMN gene and an SMN2 copy of the SMN gene, the method comprising:
adding an unprocessed human blood sample to a reaction tube comprising the second group of primers and the PCR reagents from the kit;
thermal cycling the reaction tube using PCR, the thermal cycling comprising:
a denaturation cycle at 95° C. for 3 minutes;
thirty-five annealing cycles, each annealing cycle comprising:
a first phase at 95° C. for 20 seconds;
a second phase at 60° C. for 30 seconds; and
a third phase, at 68° C. for 1 minute; and
an extension cycle at 68 ° C. for 10 minutes.
